(19) United States
(12) Patent Application Publication
(10) Pub. No.: US 2004/0102820 A1
(43) Pub. Date: May 27, 2004

(54) Implantable neurostimulator

(76) Inventors: Jaouhar Mouïne, Quebec (CA); R?eacute;jean Fontaine, Quebec (CA); Sylvain Dumont, Quebec (CA); Francois Duval, Quebec (CA)

Correspondence Address: FAY KAPLUN & MARCIN, LLP, 15O BROADWAY, SUITE 702, NEW YORK, NY, 10038, US

(21) Appl. No.: 10/450,371

(22) PCT Filed: Dec. 12, 2001
(86) PCT No.: PCT/CA01/01799

(30) Foreign Application Priority Data
Dec. 12, 2000 (CA) 2328568

(51) Int. Cl. A61N 1/18

(52) U.S. Cl. 607/48

(57) ABSTRACT

A programmable neurostimulator is described herein. The neurostimulator comprises an internal part located in a patient's body and including an implant, at least one electrode, and a communication link; and an external part, connected to the implant by the communication link, the external part including a user interface. Wherein the user interface enables programming stimulation algorithms in the implant through the communication link so that, when activated, the implant generates electrical stimulation pulses by means of the at least one electrode located at sites of stimulation of the body.

### FIELD OF THE INVENTION

The present invention relates to neurostimulation aiming at correcting disorders of neurological origin. More specifically, the present invention is concerned with an electronic implant, which is inserted in a patient's body.

### BACKGROUND OF THE INVENTION

The concept of artificially stimulating the nerves of the body is known in the art.

For example, U.S. Pat. No. 3,870,051, issued to Brinley, discloses a system of urinary control, based on stimulating selected nervous regions of the body by means of implants. Since such implants are not self-powered, nor provided with integrated intelligence, they cannot be used by themselves. The patient needs to wear a belt holding a battery and an electric stimulator.

Recently, Medtronic Inc. designed a controler implant called Interstim, embodied in ITREL II or ITREL III. This type of implant may be used for urinary control and are provided with an integrated intelligence by way of an integrated circuit, developed by Medtronic Inc.

The systems proposed by Brinley and by Medtronic Inc. share common features including the following: they both use a voltage source in order to generate bipolar pulses, according to a single algorithm. Both are devoid of external alarm. However, Medtronic's implant has an autonomy comprised between 3 and 5 years, and is provided with an encapsulating shell made of titanium, whereas Brindley's device, provided with a silastic capsule, is not to be implanted.

### OBJECTS OF THE INVENTION

The general object of the present invention is to provide an improved programmable neurostimulator.

### SUMMARY OF THE INVENTION

More specifically, in accordance with the present invention, there is provided a programmable neurostimulator comprising:

an internal part located in a patient's body and including an implant, at least one electrode, and a communication link;

an external part, said external part being connected to said implant by said communication link, said external part including a user interface; wherein said user interface enables programming stimulation algorithms in said implant through said communication link so that, when activated, said implant generates electrical stimulation pulses by means of said at least one electrode located at sites of stimulation of said body.

According to another aspect of the present invention, there is provided a programmable device for urinary control comprising an implantable part and an external part, said internal part being able to implement a plurality of stimulation algorithms of different modes and following a sequence, and to stimulate a plurality of stimulation sites corresponding to a plurality of electrodes.

According to another aspect of the present invention, there is provided a programmable device comprising:

a internal part, implantable in the body of a patient, said internal part including means for generating a train of electric pulses having a programmable width, amplitude and period; said internal part also including at least one stimulation generating means to transmit said train of electric pulses to the body of the patient;

an external part for programming and controlling said internal part.

According to yet another aspect of the present invention, there is provided a neurostimulation method comprising the acts of:

providing at least one electrode in a patient's body

providing an implant connected to the at least one electrode;

configuring said implant to generate a train of electrical pulses having a programmable amplitude, width and period according to a predetermined algorithm;

providing an interface enabling a health care specialist to program the implant.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a block diagram of an implant system, according to one embodiment of the present invention;

FIG. 2 is a block diagram an implant of the system of FIG. 1;

FIG. 3 is an illustration of conventional and random stimuli; and

FIG. 4 is an illustration of conventional and progressive stimuli.

### DESCRIPTION OF THE EMBODIMENT

Generally stated, the present invention is concerned with a neurostimulator for use by people whose nervous system is defective, causing defective physiological functions, such as urinary incontinence.

More specifically, the underlying principle is to insert electrodes in the region of the nerve that is involved in the defective function, so as to generate electrical stimulation for artificially monitoring the nerve response.

In particular, in the case of urinary incontinence for example, electrodes are inserted in the region of the sacred foramen, located at the bottom end of the backbone, so as to generate electrical pulse trains that monitor urination by coordinating the relative reflex activity of the bladder, the sphincter and the pelvis. When feeling a need to urinate, the patient presses a button on an external miniaturized remote control device. Thus, through a transcutaneaous communication link, a signal is conveyed to the implant, allowing urination by ending urine retention.

Depending on the type of application, a well-defined stimulation is needed to match the patient's pathological condition.

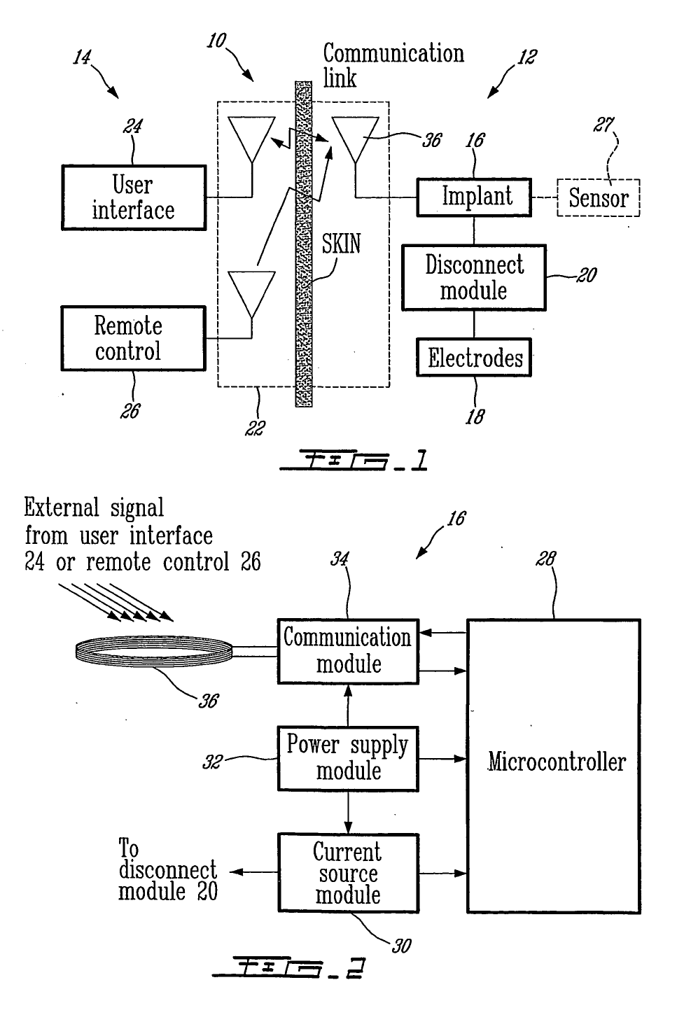

An implant system 10 according to an embodiment of the present invention will now be described with reference to FIG. 1.

The implant system 10 comprises an internal part 12 and an external part 14.

The internal part 12 includes an implant 16, a number of electrodes 18, a disconnect module 20, and a communication link 22.

The implant 16 is responsible for the generation of electrical stimulation pulses. The electrodes 18 are located at precise sites of stimulation so as to deliver the electrical stimulation pulses to the nerve. The disconnect module 20 is used to electrically connect the electrodes 18 to the implant 16 in a way that allows changing the implant 16 without removing the electrodes 18, thus reducing the risks of damaging the connected nerve.

The communication link 22 connects the implant 16 in the internal part 12 to the external part 14.

The external part 14 includes a user interface 24 and a remote control 26.

The user interface 24, which is incorporated in a computer, enables a health care specialist to program, adjust and monitor the stimulation parameters so as to achieve an appropriate stimulation algorithm. This programming of the implant is done through the communication link 22.

The remote control 26, which is reduced in size, enables the patient to control the implant, by switching on and off the stimulation, as described hereinabove, through the communication link 22.

It is to be noted that while the communication link 22 is shown herein as using antennas, other communication links could be used, such as, for example, infrared or magnetic links.

Turning now to FIG. 2, the implant 16 will now be described in further details.

The implant 16 includes a central processor or microcontroller 28 to which are connected a current sources module 30, a power supply module 32 and a communication module 34, for communication with the external part 14.

The microcontroller 28 is provided with software that enables it to support desired features, such as stimulation algorithms, memorization and data reading. It is the core of the implant 16 since it monitors all the operations of the system. Of course, the microcontroller 28 may be a custom item or it can be a simple commercial processor or microcontroller 68HCII from Motorola or Pic16CXXX from Microchip.

As a specific feature of the present invention, this central part is endowed with intelligence dedicated to neurostimulation, and is provided with a high degree of versatility and programmability. The software used is similar to that of an operating system of a computer, which enables easy programming and easy up-dating of any stimulation algorithm, together with the desired parameters and specifications, while requiring very reduced memory. It is also capable of monitoring communication in a bi-directional fashion with the external part 14 by means of appropriate interfaces. As will be apparent to one skilled in the art, such an intelligent microcontroller may be used in a range of neurostimulators besides urinary implants.

More precisely, the software is made of two parts. Firstly, a master software, which is recorded in the ROM of the microcontroller, monitors the sequence of operations of the system. Additionally, this master software manages data by executing different input/output commands, and stores a detailed description of the sequences of steps involved in the execution of macrocommands that are used by the clinician when designing a stimulation algorithm. Secondly, a stimulation program is stored in the RAM of the implant, as designed by the health care professional with the help of macrocommands describing the stimulation through adequate parameters such as, for example, stimulation energy; electrode delay; stop all stimulations.

The current sources of the module 30 are the sources of the stimulation train of pulses. They are controlled by the microcontroller 28 and are able to inject a precise amount of charges on the electrodes 18 from the power supply module 32. Moreover, they are able to generate stimulation according to different stimulation modes, namely monopolar mode, wherein a electrode acts as a source with a current return via a ground located relatively far from the electrode; bipolar mode, wherein an electrode acts as a source whereas another electrode acts as a return; or multipolar mode, wherein one or several electrodes are sources while one or several electrodes are return electrodes.

The power supply 32 includes a battery (not shown) and supplies the microcontroller 28, the current source module 30 and the communication module 34.

The communication module 34, controlled by the microcontroller 28, includes a bi-directional antenna 36 that may receive signal from the user interface 24 or from the remote control 26 and that can send signal to the user interface 24. The communication module is so configured as to extract the information provided through the communication link 22 from the external part 14 and to participate in the programming of the system and in the preparation of the data to be sent to the external part 14 on reading internal data.

FIG. 1 also shows an optional sensor 27 connected to the implant. This sensor may be used, for example for bladder sensing to know if it is full, or for nerve sensing to monitor activities on which decisions can be taken.

A feature of the present system is that a plurality of stimulation algorithms may simultaneously be stored in memory, which enables obtaining better results and possibly power saving. For example, the present system may offer a standard stimulation algorithm, encountered in conventional systems, though using it with any of the three above-mentioned stimulation modes and using stimulation trains on a plurality of simultaneous sites or according to a desired sequence. This, in turn, opens the way to using a two-ways stimulation, consisting in involving both sides of the nervous system of the human body. The stimulations could be on the right side of the spinal cord or on the left side or both which increase the possibilities of obtaining efficient stimulation sites.

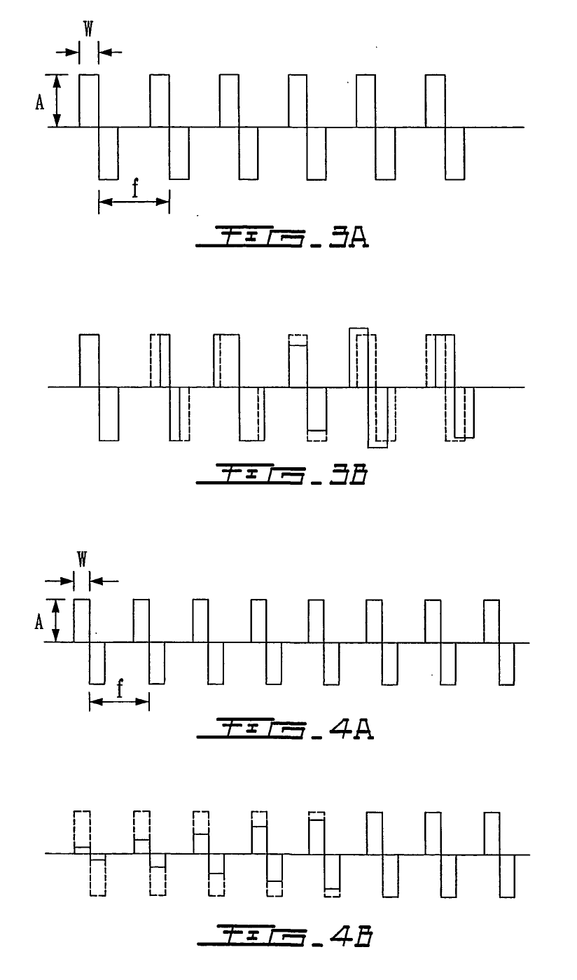

A first possible algorithm is illustrated in FIG. 3. FIG. 3a illustrates a conventional stimulus impulsion train, encountered in conventional systems, where all the impulsions have the same duration (W), period (f) and amplitude (A).

FIG. 3b illustrates an example of an algorithm according to an aspect of the present invention, which is essentially the conventional stimulation algorithm of FIG. 3a, improved by using an electrical train of pulses of random amplitude and/or frequency and/or width by interval, while keeping a predetermined average. In fact, these parameters define the amount of charges that are delivered to the nerve. By so varying them, a way is provided to prevent the nerve from getting accustomed, and thus less responsive, to the specific electrical stimulation.

A second possible algorithm is presented in FIG. 4. FIG. 4a is similar to FIG. 3a that illustrates a conventional impulsion train.

The algorithm of FIG. 4b is intended for use specifically in a urinary implant. It involves delivering stimulation in a progressive fashion to the nerve. Generally speaking, when empty, the bladder only exerts a weak pressure on the urine it contains, and does not need to be strongly stimulated to hold the urine. Therefore, the idea is, once the bladder is emptied, to start over the stimulation sequence so that the amount of charges increases progressively. In practice, this kind of algorithm monitors an electrical train of pulses in which the amplitude of each pulse is higher than that of the previous pulse. Such a process allows saving power, and thus increases the life span of the battery. It is to be understood that this algorithm can be provided with random features illustrated in FIG. 3b.

Additionally, it is contemplated that various circumstances can influence the amount of charges that is necessary for the system to be efficient. Special features enable the patient to control the level of stimulation within a range that is pre-programmed by the health care specialist, thus ensuring the efficiency of the implant while increasing considerably the life span of the battery. For instance, in the case of a urinary implant, there is less pressure exerted on the bladder at night or generally in times of rest when the body is still, so that fewer efforts are needed to hold urine. More globally, the efforts deployed for holding urine vary depending on the state of activity of the patient.

It is to be understood that the previous algorithms were described by way of examples and that other algorithms can be implemented in the implant 16.

We will now describe in more details the set of electrodes 18. These electrodes give access to a plurality of possible stimulation sites. They may vary in number, for example between 1 and 4, each electrode being able to stimulate at least 4 neighboring independent sites.

Usually, depending on the application, there are several specific stimulation sites in relation to their location versus the nerves, and the depth of insertion of each electrode depends on the patient's anatomy. However, the exact location of the stimulation is generally not precisely known. Therefore, selecting a plurality of neighboring and independent sites increases the probability of locating an electrode at a site where a maximum response of the target nerve can be obtained. Furthermore, providing a plurality of electrodes enables to perform two-ways stimulation, i.e. on both sides of the spinal cord of the human body, as is the case in a healthy urinary system, so that the performances of the stimulating system are greatly improved. Additionally, this allows the use of more advanced stimulation algorithms for activating more than one stimulation site with a predetermined time synchronization.

In an embodiment, the implant of the present invention is provided with 16 stimulation sites distributed among a maximum of 4 electrodes. Such an increased number of stimulation sites, from 4 to 16 in this example, has important effects. In particular, in the case of urinary implants, since electrodes are inserted in the sacred vertebra, it can happen that the big toe is stimulated, meaning that the related stimulation site is mistaken, so that only three sites are left for activating the adequate nerve. The implant of the present invention then provides probabilities four times higher to hit efficient stimulation sites, thus increasing the probability of success of the implant and decreasing the risk of post-implantation urinary leaks.

Moreover, the stimulation sites need be renewed approximately every 6 months in order to prevent degradation of the myelin coating of the nerve after a prolonged time of being stimulated. In an implant having only 4 stimulation sites, usually all located on the same region of the nerve, which can be alternatively stimulated, the nerve soon gets damaged. The possibility to use 16 sites in the implant of the present invention permits rotation of the stimulation loci on a longer period of time, leaving time for the myelin coating to grow again around the nerve.

Additionally, when the electrodes 18 are in place in the body, biological tissues grow on their surfaces, and it is consequently difficult to remove them without damaging the cells around, when the internal independent battery powering the implant needs to be changed. To solve this problem, the internal part 12 is provided with a disconnect module 20, which is designed so as to enable the removable electrical connection between the implant 16 to the electrodes 18 in order to allow the replacement of the implant 16 without removing the electrodes 18 from their site.

As mentioned hereinabove, a health care specialist programs the implant, and designs a stimulation algorithm that is stored in the available RAM of the microcontroller 28. Thereafter, the patient is able to control the implant in order to urinate. To permit such features, the present system is provided with a communication link 22 between the internal part 12 and the external part 14. It is essentially an inductive link that enables a serial communication across the skin. When the patient controls the implant, the communication takes place unidirectionally between the remote control 26 and the implant 16. In times of clinical programming, a two-ways communication allows the health care specialist to validate the data contained in the dedicated memory of the implant.

For programming the operations and for adjusting the stimulation parameters, the implant is provided with an expert system to be used by a health care specialist. It is essentially a user-friendly piece of software, for example developed on an IBM compatible personal computer that does not require any specific training. The software allows to select a stimulation algorithm and to set up the stimulation parameters in a graphical and interactive way. Then the algorithm may be transferred to the implant 16 via the communication link 22.

As mentioned hereinabove, the patient controls the implant by means of the remote control device 26. This device also enables the patient to monitor the level of stimulation required depending to the patient's activities, in accordance to the fine tune-up made by the health care specialist.

Optionally, in applications requiring periodic check-ups, the system may be provided with an alarm. Such an alarm may be pre-set either by the health care specialist or by the patient to a desired time. It is used to remind the patient that it is time to trigger the stimulation. The alarm signal may be acoustic, visual or of the touch-sensitive type.

As for the package, the implant 16 is encapsulated hermetically in a case made of titanium or in other biocompatible material, and provided with the required contacts for the electrical connection of the electrodes.

It is also to be noted that even though the embodiment described herein uses a remote control to control the implant, other controlling mechanisms could be used, depending on the intended use of the implant.

As may be apparent from the above disclosure, the system of the present invention is versatile, completely programmable and user-friendly.

Although the present invention has been described hereinabove by way of specific embodiments thereof, it can be modified, without departing from the spirit and nature of the subject invention as defined in the appended claims.

What is claimed is:

1. A programmable neurostimulator comprising:
an internal part located in a patient's body and including an implant, at least one electrode, and a communication link;
an external part, said external part being connected to said implant by said communication link, said external part including a user interface;
wherein said user interface enables programming stimulation algorithms in said implant through said communication link so that, when activated, said implant generates electrical stimulation pulses by means of said at least one electrode located at sites of stimulation of said body.

2. A programmable neurostimulator according to claim 1, wherein said stimulations are generated in a plurality of modes selected in the group consisting of monopolar mode, bipolar mode, multipolar mode and in a sequence.

3. A programmable neurostimulator according to claim 1, wherein said stimulations are generated following an algorithm using pulses which can have random amplitude and random frequency and random width by interval, in such a way as to obtain a desired amount of charges that are delivered to a nerve of said patient's body.

4. A programmable neurostimulator according to claim 1, wherein said stimulations are generated following an algorithm that enables delivering stimulation in a progressive amplitude to said patient's body, so that an increasing amount of charges is delivered to the patient.

5. A programmable neurostimulator according to claim 1, wherein said internal part further comprises a disconnect module removably connecting said at least one electrode to said implant.

6. A programmable neurostimulator according to claim 1, wherein said at one least one electrode provides at least 4 stimulation sites.

7. A programmable neurostimulator according to claim 1, wherein said external part further comprises a remote control enabling the user to control the operation of the implant.

8. A programmable neurostimulator according to claim 1, wherein said external part further comprises an alarm.

9. A programmable neurostimulator according to claim 1, wherein said user interface is incorporated in a computer.

10. A programmable neurostimulator according to claim 1, wherein said stimulation algorithms allows random stimulation on a plurality of stimulation sites.

11. A programmable neurostimulator according to claim 1, wherein said implant comprises:
a microcontroller supporting a software allowing programming and updating a variety of stimulation algorithms and stimulation parameters; and
a current source module generating a stimulation train of pulses; said current sources module being connected to said electrode;
wherein said microcontroller monitors said current source module so that said current source module generates a controlled amount of charges according to different stimulation modes in the group including monopolar, bipolar and multipolar modes.

12. A progammable neurostimulator according to claim 11, wherein said microcontroller is selected from the group consisting of microprocessors and application specific integrated circuits (ASIC).

13. A programmable device for urinary control comprising an implantable part and an external part, said internal part being able to implement a plurality of stimulation algorithms of different modes and following a sequence, and to stimulate a plurality of stimulation sites corresponding to a plurality of electrodes.

14. A programmable device according to claim 13, wherein said implantable part is inserted in a region of the sacred foramen.

15. A programmable device according to claim 13, wherein said external part comprises:
a user interface to program the stimulation algorithms in said internal part; and
a remote control to control said internal part.

16. A programmable device according to claim 13, wherein said stimulations are of a mode comprised in the group consisting of monopolar, bipolar and multipolar stimulation modes.

17. A programmable device according to claim 13, wherein said plurality of electrodes comprises four electrodes, and said plurality of stimulation sites comprises four stimulation sites for each said four electrodes.

18. A programmable device comprising:
a internal part, implantable in the body of a patient, said internal part including means for generating a train of electric pulses having a programmable width, amplitude and period; said internal part also including at least one stimulation generating means to transmit said train of electric pulses to the body of the patient;
an external part for programming and controlling said internal part.

19. A neurostimulation method comprising the acts of:
providing at least one electrode in a patient's body
providing an implant connected to the at least one electrode;
configuring said implant to generate a train of electrical pulses having a programmable amplitude, width and period according to a predetermined algorithm;
providing an interface enabling a health care specialist to program the implant.

20. A neurostimulation method according to claim 19, wherein the algorithm is such that the amplitude of each pulse of the electrical train of pulses is higher than the previous pulse.

21. A neurostimulation method according to claim 19, wherein the algorithm is such that the amplitude, width and period of each pulse of said electrical train of pulses varies randomly while keeping a predetermined average.
